(19) United States
(12) Patent Application Publication
(10) Pub. No.: US 2019/0328929 A1
(43) Pub. Date: Oct. 31, 2019

(54) SPINAL CAGE AND METHODS OF MANUFACTURING THE SAME

(71) Applicant: SABIC Global Technologies B.V., Bergen Op Zoom (NL)

(72) Inventors: Andrew KUGLER, Albany, NY (US); Lynn COLUCCI-MIZENKO, Niskayuna, NY (US)

(21) Appl. No.: 16/310,676

(22) PCT Filed: Jun. 21, 2017
(86) PCT No.: PCT/US2017/038473

§ 371 Date: Dec. 17, 2018

Related U.S. Application Data
(60) Provisional application No. 62/352,732, filed on Jun. 21, 2016.

(51) Int. Cl. A61L 27/18; A61L 27/54; A61F 2/44; A61F 2/30; A61L 27/44; B33Y 50/02; B29B 11/08; B33Y 80/00; B33Y 70/00; B29C 64/393

(52) U.S. Cl. CPC A61L 27/18; B33Y 10/00; A61F 2/447; A61F 2/30749; A61F 2/30942; A61L 27/446; A61L 27/443; B33Y 50/02; B29B 11/08; B33Y 80/00; B33Y 70/00; B29C 64/393; A61F 2002/30719; A61F 2002/30953; A61F 2002/30985; A61F 2002/30065; A61F 2002/30153; A61F 2002/30677; A61L 2430/38; A61L 2300/404; A61L 2300/406; A61L 2300/408; A61L 27/54

(57) ABSTRACT

Devices prepared from resins are disclosed. In one aspect, a spinal cage is disclosed for implantation between two adjacent vertebrae, the spinal cage formed from a polymer composition comprising a polyetherimide, polyether ether ketone or other biocompatible resin, the spinal cage formed from a process comprising: receiving an input relating to design specifications of the spinal cage; and causing formation of at least a portion of the spinal cage based upon the input and using one or more of an additive and subtractive process.

### TECHNICAL FIELD

The disclosure generally relates to implantable medical devices and surgical instruments having improved properties, and more particularly to a spinal fusion system, including a spinal cage having improved mechanical strength and biocompatibility while promoting fusion between vertebrae, and methods of making the same.

### BACKGROUND

Intervertebral disc degeneration is a common problem increasingly suffered by many people. Typically, this spinal problem has been addressed by removing the damaged or defective disc material and replacing it with a spinal implant which fuses two adjacent vertebrae.

Spinal fusion techniques, such as interbody fusion, involve placing a bone graft between the vertebrae in the area occupied by the intervertebral disc. The damaged disc is removed entirely in preparation for the spinal fusion. A spinal cage is then placed between the vertebrae to maintain spine alignment and disc height. Spinal fusion then takes place between the end plates of the vertebrae. Spinal fusion systems consist of a spinal cage positioned between two adjacent vertebrae to facilitate spinal fusion. The spinal fusion system also includes a rod or a plate that is connected to two adjacent vertebrae, to obtain fixation of the vertebrae with respect to each other, and can consist of a combination of both a spinal fusion cage and a rod or a plate. Insertion tools and other surgical instruments specially designed for the spinal fusion system are used to secure the spinal cage to the vertebrae.

In view of the structural integrity requirements of these implantable medical devices, the materials of fabrication are limited, and conventionally include various metal, plastic and composites. Spinal fusion systems are usually composed of metals, such as titanium or cobalt chrome alloys, or from polyetheretherketone (PEEK) and PEEK compounds or blends, a polymer that is commonly used in implantable medical devices. A problem associated with implantable medical devices is infection, which may in some cases lead to sepsis and death. As a result, it is critical that implantable medical devices and the surgical instruments used to implant them are properly sterilized prior to implantation. Therefore, the devices as well as the surgical instruments must be composed of materials that are not only capable of sterilization prior to surgery, but also highly resistant to infection once they are implanted. Conventional implantable-grade or medical-grade polymeric devices, however, may be sensitive to temperature, radiation, and moisture of traditional sterilization processes.

Therefore, there is a need for an implantable medical devices that have biocompatibility, strength, flexibility, wear resistance, and radiolucency, yet do not undergo meaningful loss of structural integrity, are not discolored, and do not lose electrical properties as a result of multiple sterilizations. There is also a need for a polymeric implantable medical that is capable of being sterilized by radiation, such as gamma and E-beam sterilization procedures. Gamma and E-beam sterilization typically subjects devices to irradiation sterilization but traditional polymeric devices, in particular, will inevitably be affected by the radiation and will experience changes in their polymer structure (such as chain scission and cross-linking). These processes may lead to significant changes and compromise in the tensile strength, elongation at break, and yield strain of such polymeric devices. Furthermore, the exact changes in mechanical properties may not be immediately apparent as there can be some time delay in the development of these changes. There is a further need for a polymeric implantable medical that is MRI (magnetic resonance imaging) compatible.

Moreover, a variation of anatomy and pathology in spines exists from patient to patient. An individual patient has a specific anatomy requiring a specific implant to ensure a successful implant. There is a need for a personalized/customized implant meeting the foregoing needs of biocompatibility, strength, and resilience to sterilization. Current personalized approaches such as those solely being based on additive manufacturing, however, may result in inferior mechanical properties, in all dimensions.

Accordingly, the present disclosure provides a potential path to such customized medical devices, including spinal fusion systems and spinal cages, and surgical instruments that have improved properties over currently existing implantable medical devices and surgical instruments.

### SUMMARY

In accordance with one aspect of the disclosure, a spinal fusion system including a spinal cage is disclosed. In accordance with another aspect of the disclosure, a customized spinal cage for implantation between two adjacent vertebrae is disclosed. The customized spinal cage may be formed from a polymer composition comprising a polyetherimide. The customized spinal cage may be formed using a hybrid technique, whereby a core is formed using injection molding and customization is implemented using a second technique such as additive or subtractive manufacturing.

In further aspects of the present disclosure, a spinal cage for implantation between two adjacent vertebrae is disclosed. The spinal cage may be formed from a polymer composition. The spinal cage may be formed from a process comprising: receiving an input relating to design specifications of a standardized spinal cage; and causing formation of at least a portion of the spinal cage based upon the input and using an additive and a subtractive process on a spinal cage core. The spinal cage core may be a standardized pre-manufactured spinal cage core.

### DETAILED DESCRIPTION

Before the present methods and devices are disclosed and described, it is to be understood that the methods and devices are not limited to specific synthetic methods, specific components, or to particular compositions. It is also to be understood that the terminology used herein is for the purpose of describing particular aspects only and is not intended to be limiting.

As used in the specification and the appended claims, the singular forms “a,” “an” and “the” include plural referents unless the context clearly dictates otherwise. Ranges can be expressed herein as from one value (first value) to another value (second value). When such a range is expressed, the range includes in some aspects one or both of the first value and the second value.

Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

It is to be understood that the terminology used herein is for the purpose of describing particular aspects only and is not intended to be limiting. As used in the specification and in the claims, the term “comprising” can include the embodiments “consisting of” and “consisting essentially of.” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific aspect or combination of aspects of the disclosed methods.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

In certain aspects of the present disclosure, implantable medical devices having improved mechanical strength and biocompatibility while promoting fusion between vertebrae are disclosed based on laboratory testing as stated in this patent application.

### The Spinal Cage

In accordance with one aspect of the disclosure, a spinal fusion system including a spinal cage is disclosed. The spinal fusion system may be used in a spinal fusion surgery. Various spinal fusion surgeries and techniques are contemplated by this disclosure, including but not limited to Posterior Lumbar Interbody Fusion (PLIF), Transforaminal Lumbar Interbody Fusion (TLIF), Anterior Lumbar Interbody Fusion (ALIF) and extreme lateral interbody fusion. The spinal fusion system includes a spinal cage. In one aspect, the spinal cage is provided for implantation between adjacent vertebrae in spaced relation while promoting interbody bone ingrowth and fusion. The spinal fusion system of the present disclosure may meet current needs of addressing variation in spinal anatomy and pathology among individual patients. The disclosed spinal fusion system may combine both additive and subtractive manufacturing techniques. The spinal fusion system may provide a personalized or customized implant that exhibits both desirable mechanical and physical properties and may encourage bone in-growth to the intervertebral structure ensuring implant success. The disclosed systems may thus improve upon conventional implants formed by only additive manufacture.

The spinal fusion system may be considered a hybrid system as the desired spinal fusion system may begin with a blank or standardized spinal cage. The “blank” or standardized spinal cage may refer to a pre-manufactured spinal cage that does not include a personalized feature. The standardized spinal cage may include common geometry/dimensions and robust mechanicals. The standardized spinal cage may refer to a general spinal cage implant that has been molded (via injection molding for example). A process of forming the disclosed personalized spinal fusion system may thus begin with a standardized spinal cage. The standardized spinal cage may be customized via personalization processes, which may be additive or subtractive or a hybrid. For example, the personalization processes may include machining (subtractive) or three-dimensional (3D) printing (additive) to impart patient-specific features to the standardized spinal cage.

A human spine includes multiple vertebrae with intervertebral spaces containing discs of the spine. The discs may become ruptured by injury or weakened by disease or degeneration, as illustrated by the defects shown in the top disc. As a surgical treatment, a spinal cage may be inserted within the affected intervertebral space for the purpose of fusing two or more vertebrae together. Spinal fusion may be used where one or more spinal discs have degenerated or ruptured recurrently. As is common practice, spinal cages may be inserted into the spine through various procedures commonly known as ALIF, PLIF, and TLIF procedures. To accomplish the goal of fusing certain vertebrae of the spine, the spinal cages described herein may be installed with bone cement, a demineralized bone matrix, and/or other bone growth agents in order to facilitate fusion of the vertebrae. Although these bone growth agents may be included in many of the described techniques and may be used with the described spinal cages, the details of this use of bone growth agents is not described herein in order to focus on the inventive aspects of the spinal cage that are the subject of this disclosure.

The spinal cage may include a body that approximates the shape and size of the annulus portion of a disk which normally separates two vertebral bodies. In one aspect of the disclosure, the spinal cage may have a generally rectangular body. The rectangular body may be tapered. In one aspect of the disclosure, the rectangular body may have curved surfaces to anatomically match the curvature of the “normal” or average vertebrae. The rectangular body may also include ridges that further serve to hold the spinal cage in place. The ridges may also reduce the possibility of the spinal cage sliding in any direction along the end plates and to prevent rotation of the spinal cage.

In certain aspects, a body or core (e.g., blank, plug, form, etc.) of a spinal cage may be formed using a first method such as injection molding. The core may include any portion of the spinal cage. However, the core may be further customized for a particular patient based on patient data such as x-rays, magnetic resonance imaging (MRI), or other medical information relevant to the patient and the implementation of the spinal cage. That is, the core may be modified based upon custom data for a specific or individual patient. For example, the core may be customized through additive manufacturing to apply surface treatment or structural features (i.e., custom data) that are specific to that patient. As another example, the core may be customized through subtractive manufacturing to treat the surface of the core or remove structural portions of the core for implementation. As such, the core or body of the spinal cage may be prepared to interface with the specific geometry of a patient's vertebrae. Moreover, the custom fit of the spinal cage of the present disclosure also includes the mechanical properties of an injection molded piece.

As an illustrative example, information may be collected form a patient including information relating to the spine of the patient. Such information may be collected through image processing such as analyzing magnetic resonance imaging (MRI) data to determine the specific shape and structure needed to best fit the area of the patient's spine. Other analytics, imagining, and spatial data may be used to determine the custom design for a patient. For example, modelling techniques may be used to model the interfacing of the implantable device with various surfaces of the patient's anatomy (e.g., vertebrae). Pressure points, gaps, alignment, registration, and the like may be analyzed through the modeling to determine the best fit of the implantable device for the specific patient. Such information may be used to program an additive or substantive manufacturing device to provide a customized three-dimensional apparatus such as a spinal cage. Other implantable apparatus may also be manufactured in a similar manner.

Generally, an additive manufacturing production technology may allow for the inclusion of a patient's personalized or custom features, but these structures may suffer from lesser physical properties as compared to the hybrid approach. These disadvantages may be attributed to the using solely an additive manufacturing process; the structure may lose some integrity because of the presence of many layers (for example, dozens or hundreds of layers) rather than a single, unitary body. Structures formed from a molded “standard core” via a subtractive manufacturing process such as, machining via a mill, for example, provide good bulk physical and mechanical properties as the structure is a single body. The systems of the present disclosure provide implants achieved via a hybrid manufacturing process where the benefits of additive and/or subtractive manufacturing may both be exploited. As such, the performance properties of the molded (or machined) core may be maintained, while leveraging the customizable benefits of additive manufacturing, subtractive manufacturing, or both. In a certain aspect, instead of the entire apparatus being manufactured through additive manufacturing, the core may be injection molded (or machined, for example, by a similar subtractive process) and only a portion of the apparatus may be manufactured using the additive or subtractive manufacturing techniques or a combination approach including both techniques.

As an example, surface geometry of an apparatus/implant may be customized to match a particular patient's interfacing vertebrae. Such implant geometry may be provided by analyzing the spinal interface of the patient based on images such as MRI, modeling, X-ray, and the like. As a further example, protrusions, surface pores, registration features, and the like may be added to a molded core. As another example, detents, pores, registration features, and fine tuning of the overall shape may be provided using subtractive manufacturing techniques.

In one aspect, the spinal cage may include an insertion tool guide and engagement features, such as bores and notches. In one aspect, the spinal cage may include windows that allow the bone to grow from one vertebra through the cage and into the adjacent vertebra. In some aspects, the windows may be partially or completely filled with a bone graft and/or synthetic bone material for stimulating bone growth between the adjacent vertebra.

In one aspect, the spinal fusion system includes a plate that is mated to the spinal cage. The plate is configured to receive, retain and orient bone screws, thereby holding the spinal cage and adjacent vertebrae in a stable relationship to promote fusion.

### Polymer Composition

In one aspect of the disclosure, the spinal cage may be formed using a polymer composition. In one aspect of the present disclosure, the polymer composition comprises a thermoplastic resin. Other components, however, may also be included in the thermoplastic resin. For example, the polymer composition may also include a ceramic and a metal. In one aspect of the disclosure, the polymer composition used to form the spinal cage is MRI (magnetic resonance imaging) compatible.

In one aspect of the disclosure, the polymer composition is suitable for melt processing such that the spinal cage may be formed using a melt process and in particular, injection molding. The polymer composition may be suitable for further personalization techniques such as an additive and/or subtractive manufacturing of an injection molded body or core. In certain aspects, this body or core (e.g., blank, plug, form, etc.) may be formed using a first method such as injection molding. The core may include any portion of the spinal cage and may be prepared for use with a patient. However, the core may be further customized for a particular patient based on patient data such as x-rays, MRIs, or other medical information relevant to the patient and the implementation of the spinal cage.

For example, the core may be customized through additive manufacturing to apply surface treatment or structural features that are specific to the patient. As such, the polymer composition may be suitable for additive manufacturing techniques. As another example, the core may be customized through subtractive manufacturing to treat the surface of the core or remove structural portions of the core for implementation. As such, the polymer composition may be suitable for subtractive manufacturing techniques. Using a hybrid manufacturing such as injection molding/additive manufacturing or injection molding/subtractive manufacturing, the core or body of the spinal cage may be prepared from to interface with the specific geometry of a patient's vertebrae. Moreover, the custom fit of the spinal cage of the present disclosure also includes the mechanical properties of an injection molded piece. As discussed herein, such properties are superior to the properties exhibited by an apparatus formed completely by additive manufacturing, for example.

The polymer composition may include any polymeric material known in the art. The polymer composition may be composed of more than one polymeric material.

In one aspect of the disclosure, the polymers used in the polymer composition may be selected from a wide variety of thermoplastic polymers, and blends of thermoplastic polymers. The polymer composition can comprise a homopolymer, a copolymer such as a star block copolymer, a graft copolymer, an alternating block copolymer or a random copolymer, ionomer, dendrimer, or a combination comprising at least one of the foregoing. The polymer composition may also be a blend of polymers, copolymers, terpolymers, or the like, or a combination comprising at least one of the foregoing.

Examples of thermoplastic polymers that can be used in the polymer composition include polyacetals, polyacrylics, polycarbonates, polyalkyds, polystyrenes, polyolefins, polyesters, polyamides, polyaramides, polyamideimides, polyarylates, polyurethanes, epoxies, phenolics, silicones, polyarylsulfones, polyethersulfones, polyphenylene sulfides, polysulfones, polyarylsulphones, polyimides, polyetherimides, polytetrafluoroethylenes, polyetherketones, polyether etherketones, polyether ketone ketones, polybenzoxazoles, polyoxadiazoles, polybenzothiazinophenothiazines, polybenzothiazoles, polypyrazinoquinoxalines, polypyromellitimides, polyquinoxalines, polybenzimidazoles, polyoxindoles, polyoxoisoindolines, polydioxoisoindolines, polytriazines, polypyridazines, polypiperazines, polypyridines, polypiperidines, polytriazoles, polypyrazoles, polycarboranes, polyoxabicyclononanes, polydibenzofurans, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, polypropylenes, polyethylenes, polyethylene terephthalates, polyvinylidene fluorides, polysiloxanes, or the like, or a combination comprising at least one of the foregoing thermoplastic polymers.

In various aspects, the polymer composition may comprise a biocompatible polymer. A biocompatible polymer may refer to a polymer composition that may be compatible with a biological organism. These polymers may be synthetic or naturally occurring polymers. Biocompatible polymers may function or interact with biological systems or organisms and thus may be tolerated by a living organism. Such biocompatible polymers may be used to replace part of a living system or to function in intimate contact with living tissue. These biocompatible polymers may include a thermoplastic polymer as described herein and/or as known in the art as biocompatible. Biocompatible polymers may include, but are not limited to, certain polyetherimides, polypropylene, polyamides, polyether ether ketones, polyether ketone ketones (PEKK), polycarbonates, polyesters, and polyether-based polyurethanes, polyarylsulphones, among others described herein. Biocompatibility of a given polymer may be assessed or confirmed according to a number of tests and may be evaluated based upon the class of device (e.g., spinal implant compared to neural implant). An exemplary standard includes ISO 10993-1.

Examples of blends of thermoplastic polymers that can be used in the polymer composition include acrylonitrile-butadiene-styrene/nylon, polycarbonate/acrylonitrile-butadiene-styrene, polyphenylene ether/polystyrene, polyphenylene ether/polyamide, polycarbonate/polyester, polyphenylene ether/polyolefin, or the like, or a combination comprising at least one of the foregoing.

In one aspect of the present disclosure, polymer composition may include polycarbonates, polysulfones, polyarylsulphones, polyesters, polyamides, polypropylene, or polyether ether ketone. In a further aspect, the polyimides used in the disclosed polymer composition may include polyamideimides, polyetherimides and polybenzimidazoles. In a further aspect, polyetherimides comprise melt processable polyetherimides.

In certain aspects, the spinal cage may include between 40 weight percent (wt. %) and 90 wt. % of thermoplastic polymer (or a blend thereof), or between about 40 wt. % and about 90 wt. % of thermoplastic polymer (or a blend thereof) and between 10 wt. % and 60 wt. % of a filler, or from about 10 wt. % and about 60 wt. %, by weight of the polymer component. Other formulations may be used.

### Polyetherimides

In one aspect of the disclosure, the polymer composition includes a polyetherimide. In an aspect, polyetherimides can comprise polyetherimides homopolymers (e.g., polyetherimidesulfones) and polyetherimides copolymers. The polyetherimide can be selected from (i) polyetherimidehomopolymers, e.g., polyetherimides, (ii) polyetherimide copolymers, and (iii) combinations thereof. Polyetherimides are known polymers and are sold by SABIC™ Innovative Plastics US LLC under the ULTEM™, EXTEM™, and Siltem™ brands (Trademark of SABIC™ Global Technologies B.V.).

In an aspect, the polyetherimides can be of formula (1):

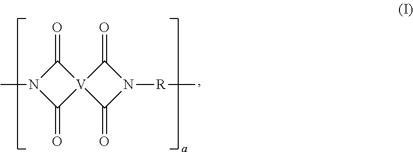

wherein a is more than 1, for example 10 to 1,000 or more, or more specifically 10 to 500.

The group V in formula (1) is a tetravalent linker containing an ether group (a “polyetherimide” as used herein) or a combination of an ether groups and arylenesulfone groups (a “polyetherimidesulfone”). Such linkers include but are not limited to: (a) substituted or unsubstituted, saturated, unsaturated or aromatic monocyclic and polycyclic groups having 5 to 50 carbon atoms, optionally substituted with ether groups, arylenesulfone groups, or a combination of ether groups and arylenesulfone groups; and (b) substituted or unsubstituted, linear or branched, saturated or unsaturated alkyl groups having 1 to 30 carbon atoms and optionally substituted with ether groups or a combination of ether groups, arylenesulfone groups, and arylenesulfone groups; or combinations comprising at least one of the foregoing. Suitable additional substitutions include, but are not limited to, ethers, amides, esters, and combinations comprising at least one of the foregoing.

The R group in formula (1) includes but is not limited to substituted or unsubstituted divalent organic groups such as: (a) aromatic hydrocarbon groups having 6 to 20 carbon atoms and halogenated derivatives thereof; (b) straight or branched chain alkylene groups having 2 to 20 carbon atoms; (c) cycloalkylene groups having 3 to 20 carbon atoms, or (d) divalent groups of formula (2):

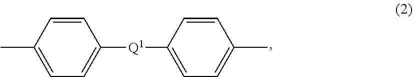

wherein Q1 includes but is not limited to a divalent moiety such as —O—, —S—, —C(O)—, —SO2-, —SO—, —CyH2y- (y being an integer from 1 to 5), and halogenated derivatives thereof, including perfluoroalkylene groups.

In an aspect, linkers V include but are not limited to tetravalent aromatic groups of formula (3):

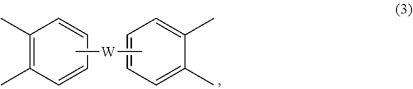

wherein W is a divalent moiety including —O—, —SO2-, or a group of the formula —O—Z—O— wherein the divalent bonds of the —O— or the —O—Z—O— group are in the 3,3′, 3,4′, 4,3′, or the 4,4′ positions, and wherein Z includes, but is not limited, to divalent groups of formulas (4):

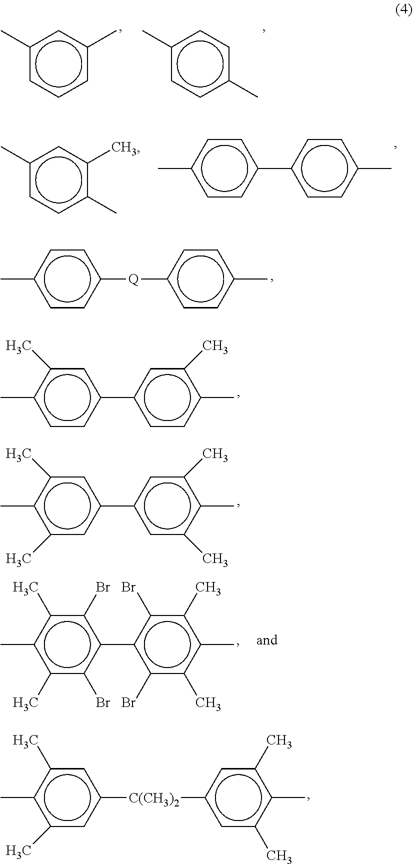

wherein Q includes, but is not limited to a divalent moiety including —O—, —S—, —C(O), —SO₂—, —SO—, —C_{y}H_{2y}— (y being an integer from 1 to 5), and halogenated derivatives thereof, including perfluoroalkylene groups.

In an aspect, the polyetherimide comprise more than 1, specifically 10 to 1,000, or more specifically, 10 to 500 structural units, of formula (5):

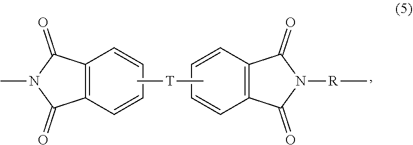

wherein T is —O— or a group of the formula —O—Z—O— wherein the divalent bonds of the —O— or the —O—Z—O— group are in the 3,3′, 3,4′, 4,3′, or the 4,4′ positions; Z is a divalent group of formula (3) as defined above; and R is a divalent group of formula (2) as defined above.

In another aspect, the polyetherimidesulfones are polyetherimides comprising ether groups and sulfone groups wherein at least 50 mole % of the linkers V and the groups R in formula (1) comprise a divalent arylenesulfone group. For example, all linkers V, but no groups R, can contain an arylenesulfone group; or all groups R but no linkers V can contain an arylenesulfone group; or an arylenesulfone can be present in some fraction of the linkers V and R groups, provided that the total mole fraction of V and R groups containing an aryl sulfone group is greater than or equal to 50 mole %.

Even more specifically, polyetherimidesulfones can comprise more than 1, specifically 10 to 1,000, or more specifically, 10 to 500 structural units of formula (6):

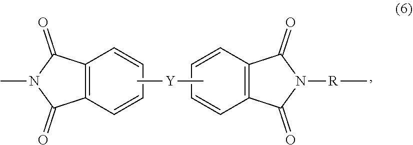

wherein Y is —O—, —SO2-, or a group of the formula —O—Z—O— wherein the divalent bonds of the —O—, SO2-, or the —O—Z—O— group are in the 3,3′, 3,4′, 4,3′, or the 4,4′ positions, wherein Z is a divalent group of formula (3) as defined above and R is a divalent group of formula (2) as defined above, provided that greater than 50 mole % of the sum of moles Y+moles R in formula (2) contain —SO₂— groups.

It is to be understood that the polyetherimides and polyetherimidesulfones can optionally comprise linkers V that do not contain ether or ether and sulfone groups, for example linkers of formula (7):

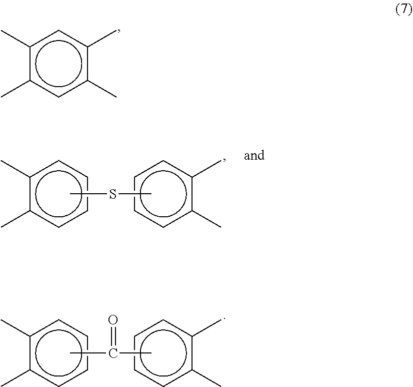

Imide units containing such linkers are generally be present in amounts ranging from 0 to 10 mole % of the total number of units, specifically 0 to 5 mole %. In one aspect no additional linkers V are present in the polyetherimides and polyetherimidesulfones.

In another aspect, the polyetherimide comprises 10 to 500 structural units of formula (5) and the polyetherimidesulfone contains 10 to 500 structural units of formula (6).

Polyetherimides and polyetherimidesulfones can be prepared by any suitable process. In one aspect, polyetherimides and polyetherimide copolymers include polycondensation polymerization processes and halo-displacement polymerization processes.

Polycondensation methods can include a method for the preparation of polyetherimides having structure (1) is referred to as the nitro-displacement process (X is nitro in formula (8)). In one example of the nitro-displacement process, N-methyl phthalimide is nitrated with 99% nitric acid to yield a mixture of N-methyl-4-nitrophthalimide (4-NPI) and N-methyl-3-nitrophthalimide (3-NPI). After purification, the mixture, containing approximately 95 parts of 4-NPI and 5 parts of 3-NPI, is reacted in toluene with the disodium salt of bisphenol-A (BPA) in the presence of a phase transfer catalyst. This reaction yields BPA-bisimide and NaNO2 in what is known as the nitro-displacement step. After purification, the BPA-bisimide is reacted with phthalic anhydride in an imide exchange reaction to afford BPA-dianhydride (BPADA), which in turn is reacted with a diamine such as meta-phenylene diamine (MPD) in ortho-dichlorobenzene in an imidization-polymerization step to afford the product polyetherimide.

Other diamines are also possible. Examples of suitable diamines include: m-phenylenediamine; p-phenylenediamine; 2,4-diaminotoluene; 2,6-diaminotoluene; m-xylylenediamine; p-xylylenediamine; benzidine; 3,3′-dimethylbenzidine; 3,3′-dimethoxybenzidine; 1,5-diaminonaphthalene; bis(4-aminophenyl)methane; bis(4-aminophenyl)propane; bis(4-aminophenyl)sulfide; bis(4-aminophenyl)sulfone; bis(4-aminophenyl)ether; 4,4′-diaminodiphenylpropane; 4,4′-diaminodiphenylmethane(4,4′-methylenedianiline); 4,4′-diaminodiphenylsulfide; 4,4′-diaminodiphenylsulfone; 4,4′-diaminodiphenylether(4,4′-oxydianiline); 1,5-diaminonaphthalene; 3,3′dimethylbenzidine; 3-methylheptamethylenediamine; 4,4-dimethylheptamethylenediamine; 2,2′,3,3′-tetrahydro-3,3,3′,3′-tetramethyl-1,1′-spirobi[1H-indene]-6,6′-diamine; 3,3′,4,4′-tetrahydro-4,4,4′,4′-tetramethyl-2,2′-spirobi[2H-1-benzo-pyran]-7,7′-diamine; 1,1′-bis[1-amino-2-methyl-4-phenyl]cyclohexane, and isomers thereof as well as mixtures and blends comprising at least one of the foregoing. In one aspect, the diamines are specifically aromatic diamines, especially m- and p-phenylenediamine and mixtures comprising at least one of the foregoing.

Suitable dianhydrides that can be used with the diamines include and are not limited to 2,2-bis[4-(3,4-dicarboxyphenoxy)phenyl]propane dianhydride; 4,4′-bis(3,4-dicarboxyphenoxy)diphenyletherdianhydride; 4,4′-bis(3,4-dicarboxyphenoxy)diphenylsulfidedianhydride; 4,4′-bis(3,4-dicarboxyphenoxy)benzophenonedianhydride; 4,4′-bis(3,4-dicarboxyphenoxy)diphenylsulfonedianhydride; 2,2-bis[4-(2,3-dicarboxyphenoxy)phenyl]propane dianhydride; 4,4′-bis(2,3-dicarboxyphenoxy)diphenyletherdianhydride; 4,4′-bis(2,3-dicarboxyphenoxy)diphenylsulfidedianhydride; 4,4′-bis(2,3-dicarboxyphenoxy)benzophenonedianhydride; 4,4′-bis(2,3-dicarboxyphenoxy)diphenylsulfonedianhydride; 4-(2,3-dicarboxyphenoxy)-4′-(3,4-dicarboxyphenoxy)diphenyl-2,2-propane dianhydride; 4-(2,3-dicarboxyphenoxy)-4′-(3,4-dicarboxyphenoxy)diphenyletherdianhydride; 4-(2,3-dicarboxyphenoxy)-4′-(3,4-dicarboxyphenoxy)diphenylsulfide dianhydride; 4-(2,3-dicarboxyphenoxy)-4′-(3,4-dicarboxyphenoxy)benzophenonedianhydride; 4-(2,3-dicarboxyphenoxy)-4′-(3,4-dicarboxyphenoxy)diphenylsulfone dianhydride; 1,3-bis(2,3-dicarboxyphenoxy)benzene dianhydride; 1,4-bis(2,3-dicarboxyphenoxy)benzene dianhydride; 1,3-bis(3,4-dicarboxyphenoxy)benzene dianhydride; 1,4-bis(3,4-dicarboxyphenoxy)benzene dianhydride; 3,3′,4,4′-diphenyl tetracarboxylicdianhydride; 3,3′,4,4′-benzophenonetetracarboxylic dianhydride; naphthalicdianhydrides, such as 2,3,6,7-naphthalic dianhydride, etc.; 3,3′,4,4′-biphenylsulphonictetracarboxylic dianhydride; 3,3′,4,4′-biphenylethertetracarboxylic dianhydride; 3,3′,4,4′-dimethyldiphenylsilanetetracarboxylic dianhydride; 4,4′-bis(3,4-dicarboxyphenoxy)diphenylsulfidedianhydride; 4,4′-bis(3,4-dicarboxyphenoxy)diphenylsulphonedianhydride; 4,4′-bis(3,4-dicarboxyphenoxy)diphenylpropanedianhydride; 3,3′,4,4′-biphenyltetracarboxylic dianhydride; bis(phthalic)phenylsulphineoxidedianhydride; p-phenylene-bis(triphenylphthalic)dianhydride; m-phenylene-bis(triphenylphthalic)dianhydride; bis(triphenylphthalic)-4,4′-diphenylether dianhydride; bis(triphenylphthalic)-4,4′-diphenylmethane dianhydride; 2,2′-bis(3,4-dicarboxyphenyl)hexafluoropropanedianhydride; 4,4′-oxydiphthalic dianhydride; pyromelliticdianhydride; 3,3′,4,4′-diphenylsulfonetetracarboxylic dianhydride; 4′,4′-bisphenol A dianhydride; hydroquinone diphthalic dianhydride; 6,6′-bis(3,4-dicarboxyphenoxy)-2,2′,3,3′-tetrahydro-3,3,3′,3′-tetramiethyl- -1,1′-spirobi[1H-indene]dianhydride; 7,7′-bis(3,4-dicarboxyphenoxy)-3,3′,4,4′-tetrahydro-4,4,4′,4′-tetraniethyl- -2,2′-spirobi[2H-1-benzopyran]dianhydride; 1,1′-bis[1-(3,4-dicarboxyphenoxy)-2-methyl-4-phenyl]cyclohexane dianhydride; 3,3′,4,4′-diphenylsulfonetetracarboxylic dianhydride; 3,3′,4,4′-diphenylsulfidetetracarboxylic dianhydride; 3,3′,4,4′-diphenylsulfoxidetetracarboxylic dianhydride; 4,4′-oxydiphthalic dianhydride; 3,4′-oxydiphthalic dianhydride; 3,3′-oxydiphthalic dianhydride; 3,3′-benzophenonetetracarboxylic dianhydride; 4,4′-carbonyldiphthalic dianhydride; 3,3′,4,4′-diphenylmethanetetracarboxylic dianhydride; 2,2-bis(4-(3,3-dicarboxyphenyl)propane dianhydride; 2,2-bis(4-(3,3-dicarboxyphenyl)hexafluoropropanedianhydride; (3,3′,4,4′-diphenyl)phenylphosphinetetracarboxylicdianhydride; (3,3′,4,4′-diphenyl)phenylphosphineoxidetetracarboxylicdianhydride; 2,2′-dichloro-3,3′,4,4′-biphenyltetracarboxylic dianhydride; 2,2′-dimethyl-3,3′,4,4′-biphenyltetracarboxylic dianhydride; 2,2′-dicyano-3,3′,4,4′-biphenyltetracarboxylic dianhydride; 2,2′-dibromo-3,3′,4,4′-biphenyltetracarboxylic dianhydride; 2,2′-diiodo-3,3′,4,4′-biphenyltetracarboxylic dianhydride; 2,2′-ditrifluoromethyl-3,3′,4,4′-biphenyltetracarboxylic dianhydride; 2,2′-bis(1-methyl-4-phenyl)-3,3′,4,4′-biphenyltetracarboxylic dianhydride; 2,2′-bis(1-trifluoromethyl-2-phenyl)-3,3′,4,4′-biphenyltetracarboxylic dianhydride; 2,2′-bis(1-trifluoromethyl-3-phenyl)-3,3′,4,4′-biphenyltetracarboxylic dianhydride; 2,2′-bis(1-trifluoromethyl-4-phenyl)-3,3′,4,4′-biphenyltetracarboxylic dianhydride; 2,2′-bis(1-phenyl-4-phenyl)-3,3′,4,4′-biphenyltetracarboxylic dianhydride; 4,4′-bisphenol A dianhydride; 3,4′-bisphenol A dianhydride; 3,3′-bisphenol A dianhydride; 3,3′,4,4′-diphenylsulfoxidetetracarboxylic dianhydride; 4,4′-carbonyldiphthalic dianhydride; 3,3′,4,4′-diphenylmethanetetracarboxylic dianhydride; 2,2′-bis(1,3-trifluoromethyl-4-phenyl)-3,3′,4,4′-biphenyltetracarboxylic dianhydride, and all isomers thereof, as well as combinations of the foregoing.

Halo-displacement polymerization methods for making polyetherimides and polyetherimidesulfones include and are not limited to, the reaction of a bis(phthalimide) for formula (8):

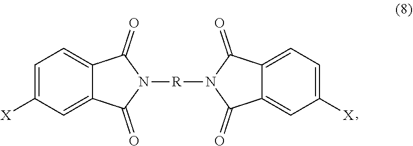

wherein R is as described above and X is a nitro group or a halogen. Bis-phthalimides (8) can be formed, for example, by the condensation of the corresponding anhydride of formula (9):

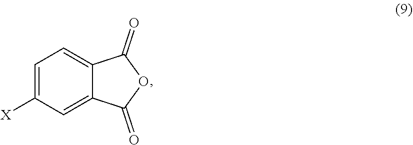

wherein X is a nitro group or halogen, with an organic diamine of the formula (10):

H₂N—R—NH₂ (10),

wherein R is as described above.

Illustrative examples of amine compounds of formula (10) include: ethylenediamine, propylenediamine, trimethylenediamine, diethylenetriamine, triethylenetetramine, hexamethylenediamine, heptamethylenediamine, octamethylenediamine, nonamethylenediamine, decamethylenediamine, 1,12-dodecanediamine, 1,18-octadecanediamine, 3-methylheptamethylenediamine, 4,4-dimethylheptamethylenediamine, 4-methylnonamethylenediamine, 5-methylnonamethylenediamine, 2,5-dimethylhexamethylenediamine, 2,5-dimethylheptamethylenediamine, 2,2-dimethylpropylenediamine, N-methyl-bis(3-aminopropyl)amine, 3-methoxyhexamethylenediamine, 1,2-bis(3-aminopropoxy) ethane, bis(3-aminopropyl) sulfide, 1,4-cyclohexanediamine, bis-(4-aminocyclohexyl) methane, m-phenylenediamine, p-phenylenediamine, 2,4-diaminotoluene, 2,6-diaminotoluene, m-xylylenediamine, p-xylylenediamine, 2-methyl-4,6-diethyl-1,3-phenylene-diamine, 5-methyl-4,6-diethyl-1,3-phenylene-diamine, benzidine, 3,3′-dimethylbenzidine, 3,3′-dimethoxybenzidine, 1,5-diaminonaphthalene, bis(4-aminophenyl) methane, bis(2-chloro-4-amino-3,5-diethylphenyl) methane, bis(4-aminophenyl) propane, 2,4-bis(b-amino-t-butyl) toluene, bis(p-b-amino-t-butylphenyl) ether, bis(p-b-methyl-o-aminophenyl)benzene, bis(p-b-methyl-o-aminopentyl)benzene, 1,3-diamino-4-isopropylbenzene, bis(4-aminophenyl) ether and 1,3-bis(3-aminopropyl)tetramethyldisiloxane. Mixtures of these amines can be used. Illustrative examples of amine compounds of formula (10) containing sulfone groups include but are not limited to, diaminodiphenylsulfone (DDS) and bis(aminophenoxy phenyl) sulfones (BAPS). Combinations comprising any of the foregoing amines can be used.

The polyetherimides can be synthesized by the reaction of the bis(phthalimide) (8) with an alkali metal salt of a dihydroxy substituted aromatic hydrocarbon of the formula HO—V—OH wherein V is as described above, in the presence or absence of phase transfer catalyst. Suitable phase transfer catalysts are disclosed in U.S. Pat. No. 5,229,482. Specifically, the dihydroxy substituted aromatic hydrocarbon a bisphenol such as bisphenol A, or a combination of an alkali metal salt of a bisphenol and an alkali metal salt of another dihydroxy substituted aromatic hydrocarbon can be used.

In one aspect, the polyetherimide comprises structural units of formula (5) wherein each R is independently p-phenylene or m-phenylene or a mixture comprising at least one of the foregoing; and T is group of the formula —O—Z—O— wherein the divalent bonds of the —O—Z—O— group are in the 3,3′ positions, and Z is 2,2-diphenylenepropane group (a bisphenol A group). Further, the polyetherimidesulfone comprises structural units of formula (6) wherein at least 50 mole % of the R groups are of formula (4) wherein Q is —SO2- and the remaining R groups are independently p-phenylene or m-phenylene or a combination comprising at least one of the foregoing; and T is group of the formula —O—Z—O— wherein the divalent bonds of the —O—Z—O— group are in the 3,3′ positions, and Z is a 2,2-diphenylenepropane group.

The polyetherimide and polyetherimidesulfone can be used alone or in combination with each other and/or other of the disclosed polymeric materials in fabricating the polymeric components of the disclosure. In one aspect, only the polyetherimide is used. In another aspect, the weight ratio of polyetherimide:polyetherimidesulfone can be from 99:1 to 50:50.

The polyetherimides can have a weight average molecular weight (Mw) of 5,000 to 100,000 grams per mole (g/mole) as measured by gel permeation chromatography (GPC). In some aspects the Mw can be 10,000 to 80,000 g/mol, or about 10,000 g/mol to about 80,000 g/mol. The molecular weights as used herein refer to the absolute weight averaged molecular weight (Mw).

The polyetherimides can have an intrinsic viscosity greater than or equal to 0.2 deciliters per gram (dl/g) as measured in m-cresol at 25° C. Within this range the intrinsic viscosity can be about 0.35 dl/g to 1.0 dl/g, as measured in m-cresol at 25° C.

The polyetherimides can have a glass transition temperature of greater than 180° C., specifically of 200° C. to 500° C., as measured using differential scanning calorimetry (DSC) per ASTM test D3418. In some aspects, the polyetherimide and, in particular, a polyetherimide has a glass transition temperature of 240° C. to 350° C.

The polyetherimides can have a melt index of 0.1 to 10 grams per minute (g/min), as measured by American Society for Testing Materials (ASTM) DI 238 at 340 to 370° C., using a 6.7 kilogram (kg) weight.

In certain aspects, the polyetherimides (PEI) of the present disclosure may be unfilled, standard flow grades (PEI-1 in Tables 1-2) or unfilled, high flow grades (PEI-2 in Tables 1-2), or may be filled, for example, with carbon (e.g., carbon fiber) or glass. Filled polymer components may include between 40 weight percent (wt. %) and 90 wt. % of the polyetherimide resin and between 10 wt. % and 60 wt. % of a filler by weight of the polymer component. Other formulations may be used.

An alternative halo-displacement polymerization process for making polyetherimides, e.g., polyetherimides having structure (1) is a process referred to as the chloro-displacement process (X is chlorine Cl in formula (8)). The chloro-displacement process is illustrated as follows: 4-chloro phthalic anhydride and meta-phenylene diamine are reacted in the presence of a catalytic amount of sodium phenyl phosphinate catalyst to produce the bischlorophthalimide of meta-phenylene diamine (CAS No. 148935-94-8). The bischlorophthalimide is then subjected to polymerization by chloro-displacement reaction with the disodium salt of BPA in the presence of a catalyst in ortho-dichlorobenzene or anisole solvent. Alternatively, mixtures of 3-chloro- and 4-chlorophthalic anhydride may be employed to provide a mixture of isomeric bischlorophthalimides which may be polymerized by chloro-displacement with BPA disodium salt as described above.

Siloxane polyetherimides can include polysiloxane/polyetherimide block or random copolymers having a siloxane content of greater than 0 and less than 40 weight percent (wt. %) based on the total weight of the block copolymer. The block copolymer comprises a siloxane block of Formula (11):

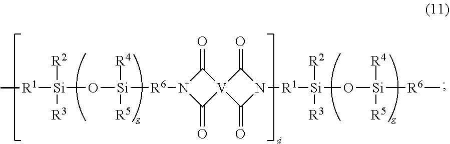

wherein R¹⁻⁶ are independently at each occurrence selected from the group consisting of substituted or unsubstituted, saturated, unsaturated, or aromatic monocyclic groups having 5 to 30 carbon atoms, substituted or unsubstituted, saturated, unsaturated, or aromatic polycyclic groups having 5 to 30 carbon atoms, substituted or unsubstituted alkyl groups having 1 to 30 carbon atoms and substituted or unsubstituted alkenyl groups having 2 to 30 carbon atoms, V is a tetravalent linker selected from the group consisting of substituted or unsubstituted, saturated, unsaturated, or aromatic monocyclic and polycyclic groups having 5 to 50 carbon atoms, substituted or unsubstituted alkyl groups having 1 to 30 carbon atoms, substituted or unsubstituted alkenyl groups having 2 to 30 carbon atoms and combinations comprising at least one of the foregoing linkers, g equals 1 to 30, and d is 2 to 20.

The polyetherimide resin can have a weight average molecular weight (Mw) within a range having a lower limit and/or an upper limit. The range can include or exclude the lower limit and/or the upper limit. The lower limit and/or upper limit can be selected from about 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 101000, 102000, 103000, 104000, 105000, 106000, 107000, 108000, 109000, and about 110000 Daltons. For example, the polyetherimide resin can have a weight average molecular weight (Mw) from 5,000 to 100,000 Daltons, from 5,000 to 80,000 Daltons, or from 5,000 to 70,000 Daltons. The primary alkyl amine modified polyetherimide will have lower molecular weight and higher melt flow than the starting, unmodified, polyetherimide.

The polyetherimide resin can be selected from the group consisting of a polyetherimide, for example as described in U.S. Pat. Nos. 3,875,116; 6,919,422 and 6,355,723 a silicone polyetherimide, for example as described in U.S. Pat. Nos. 4,690,997; 4,808,686 a polyetherimidesulfone resin, as described in U.S. Pat. No. 7,041,773 and combinations thereof, each of these patents are incorporated herein their entirety.

The polyetherimide resin can have a glass transition temperature within a range having a lower limit and/or an upper limit. The range can include or exclude the lower limit and/or the upper limit. The lower limit and/or upper limit can be selected from 100, 110, 120, 130, 140, 150, 160, 170, 180, 190, 200, 210, 220, 230, 240, 250, 260, 270, 280, 290, 300 and 310 degrees Celsius (° C.). For example, the polyetherimide resin can have a glass transition temperature (Tg) greater than about 200° C.

The polyetherimide resin can be substantially free (less than 100 parts per million parts per million (ppm), or less than about 100 ppm) of benzylic protons. The polyetherimide resin can be free of benzylic protons. The polyetherimide resin can have an amount of benzylic protons below 100 ppm. In one aspect, the amount of benzylic protons ranges from more than 0 to below 100 ppm. In another aspect, the amount of benzylic protons is not detectable.

The polyetherimide resin can be substantially free (less than 100 ppm, or less than about 100 ppm) of halogen atoms. The polyetherimide resin can be free of halogen atoms. The polyetherimide resin can have an amount of halogen atoms below 100 ppm. In one aspect, the amount of halogen atoms range from more than 0 to below 100 ppm. In another aspect, the amount of halogen atoms is not detectable.

### Therapeutic Agents

In certain aspects of the disclosure, the spinal fusion system may additionally include certain therapeutic agents that are commonly used to promote bone fusion or ingrowth. Such therapeutic agents may include natural or synthetic therapeutic agents such as bone morphogenic proteins (BMPs), growth factors, bone marrow aspirate, stem cells, progenitor cells, antibiotics, or other osteoconductive, osteoinductive, osteogenic, or any other fusion enhancing material or beneficial therapeutic agent.

In one aspect, the spinal cage includes a coating formed on surfaces of the cage. The coating, for example, may be a biomimetic and/or osteogenic (e.g., bone morphogenetic protein(s) (BMP) and related compounds) coating. In certain aspects, the coating may be used to enhance bone growth on the spinal cage. In some aspects, the coating may be formed on substantially all of the surfaces of the spinal cage; though, in other aspects, only a portion of the surfaces are coated; and, in some aspects, the spinal cage may not be coated at all. Suitable coating materials include calcium phosphate, BMP and related compounds, amongst others. In further aspects, substances designated as coating materials may be adapted and used in compounding into the polymer composition described herein.

In some aspects, a substance (e.g., a drug) may elute from the spinal cage and/or a coating on the spinal cage. For example, a substance incorporated into the spinal cage and/or coating may be emitted into regions around the implant cage (e.g., within the windows). In some aspects, the substance (e.g., BMP and related compounds) may be selected to enhance bone growth. The substance, for example, may be incorporated at different concentrations into different locations of the spinal cage and/or coating.

In certain aspects of the disclosure, the polymer composition may also include a biocide. The biocide may be selected from germicides, antimicrobials, antibiotics, antibacterials, antiyeasts, antialgals, antivirals, antifungals, antiprotozoals, antiparasites, agents promoting bone or skeletal growth, and combinations thereof.

In certain aspects of the disclosure, the spinal cage and/or the rod or plate may be formed by any method or combination of methods known in the art. These methods include, but are not limited to, molding processes, additive manufacturing, and machining. These molding processes may include, but are not limited to, various melt forming process, injection molding, profile extrusion, thermoforming, additive manufacturing, compression molding, powder sintering, transfer molding, reaction injection molding (RIM), vacuum forming, and cold casting. In one aspect, a combination of these molding methods may be used to form the spinal cage and/or the plate.

Various surgical instruments may be used to secure the spinal cage to the vertebrae. For example, a screw driver, a distractor, a reamer, a ring curette, a holder, a graft pusher, an impactor, a forked impactor, a sizer, a trial, and/or a final impactor may be used. A spinal cage may be secured to the vertebrae via anterior lumbar interbody fusion (ALIF) surgery or posterior lumbar interbody fusion (PLIF). In ALIF, the spinal cage is inserted into the body from the front of the body, such as from the abdomen, while in PLIF the spinal cage is inserted into the body from the back, such as from the lower back. For example, in ALIF, patients are positions on their backs and given an anesthesia. The surgeon may make an incision on one side of the abdomen and move the organs and blood vessels to one side to expose the front of the spine. The problem disc may be located using several means, one of which is a fluoroscope. After the problem disc has been located, the surgeon may drill two holes through the front of the disc. The spinal cage is designed to fit into the drilled holes. The spinal cage may be fitted to the drilled holes using the distractor, the reamer, the ring curette, the holder, and/or the various types of impactors. These instruments may be used on a standalone basis or multiple instruments may be used in conjunction. Bone graft material may be packed into the hollow spinal cage. Bone graft material may be bone graft from another part of the body, such as the pelvis, or it may be a bone graft substitute. The graft pusher may be used to pack the graft material into the hollow spinal cage. The surgeon may then use the screwdriver to screw the spinal cage into the holes. The threads of the spinal cage clinch the vertebrae above and below. Alternatively, instead of inserting the spinal cage into the body using one incision, multiple, smaller incisions may be used. PLIF is analogous to ALIF except that the spinal cage is inserted from the back.

In certain aspects of the disclosure, the surgical instruments may also be formed using the polymer composition disclosed herein. The implantable medical device of this or any other aspect of the disclosure may be any implant or instrument used to accomplish a medical procedure. The medical device of some aspects of the disclosure is capable of undergoing one or more sterilizations, without degrading in a manner that would make the device unsuitable for use in a medical procedure. The sterilizations may be from steam autoclave sterilization cycles or from application of a chemical sterilizing substance, or from any other effective sterilization substance or process, including, dry heat, ethylene oxide gas, vaporized hydrogen peroxide, gamma or electron beam radiation, or other sterilization procedures.

### Methods of Manufacture

In certain aspects of the disclosure, the spinal cage and/or the rod or plate may be formed by any method or combination of methods known in the art. These methods include, but are not limited to, molding processes, additive manufacturing, and machining or subtractive manufacturing. These molding processes include, but are not limited to, various melt forming process, injection molding, profile extrusion, thermoforming, additive manufacturing, compression molding, fiber extrusion, powder sintering, transfer molding, reaction injection molding (RIM), vacuum forming, and cold casting. In one aspect, a combination of these molding methods may be used to form the spinal cage and/or the plate.

In certain aspects, a core (e.g., blank, plug, form, etc.) of a spinal cage may be formed using a first method such as an injection molding (or machining via a mill, or other subtractive manufacturing process). The core may include any portion of the spinal cage and may be prepared for use with a patient. However, the core may be further customized for a particular patient based on patient data such as x-rays, MRIs, or other medical information relevant to the patient and the implementation of the spinal cage. For example, the core may be customized through additive manufacturing to apply surface treatment or structural features that are specific to the patient. As another example, the core may be customized through subtractive manufacturing to treat the surface of the core or remove structural portions of the core for implementation.

As an illustrative example, information may be collected form a patient including information relating to the spine of the patient. Such information may be collected through image processing such as analyzing MRI data to determine the specific shape and structure needed to best fit the area of the patient's spine. Other analytics, imagining, and spatial data may be used to determine the custom design for a patient. Such information may be used to program an additive or substantive manufacturing device to provide a customized three-dimensional apparatus such as a spinal cage. Other implantable apparatus may also be manufactured in a similar manner.

In certain aspect, instead of the entire apparatus being manufactured through additive manufacturing, the core may be injection molded and only a portion of the apparatus may be manufactured using the additive or subtractive manufacturing techniques. As an example, surface geometry of an apparatus/implant may be customized to match a particular patient's interfacing vertebrae. Such implant geometry may be provided by analyzing the spinal interface of the patient based on images such as MRI, modeling, X-ray, and the like. As a further example, protrusions, surface pores, registration features, and the like may be added to a molded core. As another example, detents, pores, and fine tuning of the overall shape may be provided using subtractive manufacturing techniques. As such, the performance properties of the molded core may be maintained, while leveraging the customizable benefits of additive and subtractive manufacturing.

As an illustrative example, comparative characteristics of a material (e.g., Nylon 12) are illustrated in Table 1, showing a comparison between a selective laser sintered (SLS) component and a substantially similar molded component.

TABLE 1
Characteristics of components

| | SLS | | Molded | |
|---|---|---|---|---|
| Flexural Strength | 6,850 | psi | 22,500 | psi |
| pounds per square inch (psi) | | | | |
| (ASTM D 790) | | | | |
| Heat deflection temperature HDT | 187° | F. | 325° | F. |
| at 264 psi degrees Fahrenheit (° F.) | | | | |
| (ASTM D 648) | | | | |
| Izod impact Strength (notched) | 0.8 | ft-lb/in | 2.4 | ft-lb/in |
| foot-pound per inch, ft-lb/in | | | | |
| (ASTM D 256) | | | | |
| Tensile Modulus | 246 | Kpsi | 900 | Kpsi |
| kilopounds per square inch | | | | |
| (Kpsi) (ASTM D 638/D 790) | | | | |
| Tensile Strength | 6,815 | psi | 22,500 | psi |
| psi (ASTM D 638/D790) | | | | |

As shown in Table 1, the comparative properties illustrate the improved properties such as tensile strength and tensile modulus, among others. (See, e.g., https://www.protolabs.com/resources/whitepapers/2016/materials-matter-3d-printing). As such, an apparatus that is molded may out perform the same apparatus that is formed using SLS exclusively. To maintain the improved properties, the present disclosure provides methods for manufacturing implantable devices that may include one or more manufacturing methods (e.g., hybrid manufacturing). For example, a core or blank may be formed using injection molding (or machining) and may exhibit the improved characteristics of a molded article over an SLS formed article. However, the core may be customized using additive or subtractive manufacturing of the core to exhibit the benefits of a customized implantable device.

Such a hybrid process may be used to manufacture various implantable devices, as described herein. As an example, composition including PEI may be used for the injection molded core component and the additive manufacturing aspects of the resultant apparatus. As such, the improvements exhibited by PEI over other materials such as PEEK and poly ether ketone ketone PEKK may be realized in combination with the manufacturing benefits of molding over components that are formed using only additive manufacturing.

### Aspects

The present disclosure comprises at least the following aspects.

Aspect 1. A spinal cage for implantation between two adjacent vertebrae, wherein the spinal cage comprises a polymer composition.

Aspect 2. A spinal cage for implantation between two adjacent vertebrae, wherein the spinal cage consisting essentially of a polymer composition.

Aspect 3. A spinal cage for implantation between two adjacent vertebrae, wherein the spinal cage consisting of a polymer composition.

Aspect 4. A spinal cage for implantation between two adjacent vertebrae, the spinal cage formed from a polymer composition, the spinal cage formed from a process comprising: (a) receiving an input relating to design specifications of the spinal cage; and (b) causing formation of at least a portion of the spinal cage based upon the input and using an additive manufacturing, subtractive process, or a combination thereof on a spinal cage core, wherein the spinal cage core is a standardized pre-manufactured spinal cage core.

Aspect 5. A spinal cage for implantation between two adjacent vertebrae, the spinal cage formed from a polymer composition, the spinal cage formed from a process consisting essentially of: (a) receiving an input relating to design specifications of the spinal cage; and (b) causing formation of at least a portion of the spinal cage based upon the input and using an additive manufacturing, subtractive process, or a combination thereof on a spinal cage core, wherein the spinal cage core is a standardized pre-manufactured spinal cage core.

Aspect 6. A spinal cage for implantation between two adjacent vertebrae, the spinal cage formed from a polymer composition, the spinal cage formed from a process consisting of: (a) receiving an input relating to design specifications of the spinal cage; and (b) causing formation of at least a portion of the spinal cage based upon the input and using an additive manufacturing, subtractive process, or a combination thereof on a spinal cage core, wherein the spinal cage core is a standardized pre-manufactured spinal cage core.

Aspect 7. The spinal cage of any preceding aspect, wherein the polymer composition comprises a biocompatible polymer.

Aspect 8. The spinal cage of any preceding aspect, wherein the polymer composition comprises polyetherimide, polyether ether ketone, polyether ketone ketone, polyarylsulphone, or a combination thereof.

Aspect 9. The spinal cage of any preceding aspect, wherein the polymer composition comprises a polyetherimide.

Aspect 10. The spinal cage of any preceding aspect, wherein the polymer composition comprises a polyether ether ketone.

Aspect 11. The spinal cage of any preceding aspect, wherein the polymer composition comprises a polyetherimide comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 12. The spinal cage of any preceding aspect, wherein the polyetherimide has a weight average molecular weight of at least about 10,000 to about 150,000 grams per mole (g/mol).

Aspect 13. The spinal cage of any preceding aspect, wherein the polyetherimide has less than 100 ppm amine end groups.

Aspect 14. The spinal cage of any preceding aspect, further comprising a biocide, wherein the biocide is selected from germicides, antimicrobials, antibiotics, antibacterials, antiyeasts, antialgals, antivirals, antifungals, antiprotozoals, antiparasites, agents promoting bone or skeletal growth, and combinations thereof.

Aspect 15. A medical device formed from the spinal cage of any preceding aspect, wherein the device is formed from a polymer component comprising between 40 wt % and 90 wt % of the polyetherimide resin or a polyether ether ketone resin and between 10 wt % and 60 wt % of a filler by weight of the polymer component.

Aspect 16. The medical device of aspect 11, wherein the filler comprises glass, carbon, carbon fiber, or a combination thereof.

Aspect 17. The spinal cage of any preceding aspect, wherein the polymer composition further comprises ceramic or metal.

Aspect 18. The spinal cage of any preceding aspect, wherein polyetherimide comprises repeating units of the formula

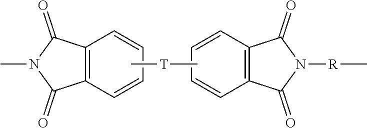

wherein R is a divalent radical of the formula

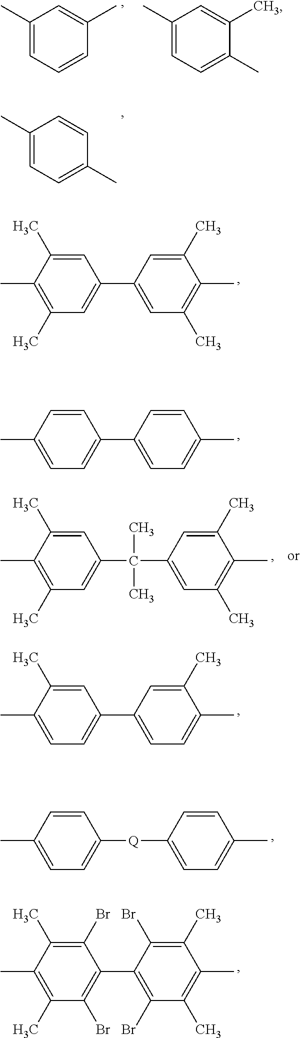

or combinations thereof wherein Q is selected from —O—, —S—, —C(O)—, —SO₂—, —SO—, and —C_{y}H_{2y}— wherein y is an integer from 1 to 5; and T is —O— or a group of the formula —O—Z—O— wherein the divalent bonds of the —O— or the —O—Z—O— group are in the 3,3′, 3,4′, 4,3′, or the 4,4′ positions and Z is a divalent group of the formula

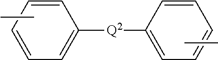

wherein Q² is selected from —O—, —S—, —C(O)—, —SO₂—, —SO—, and —C_{y}H_{2y}— wherein y is an integer from 1 to 5.

Aspect 19. The spinal cage according to any of the preceding aspects, wherein the spinal cage is sterilized using at least one sterilization process selected from the group consisting of: steam autoclave sterilization, hydrogen peroxide sterilization, gamma-ray sterilization, electron beam sterilization, and ethylene oxide sterilization.

Aspect 20. The spinal cage according to any of the preceding aspects, wherein the spinal cage has a compressive strength after sterilization that is within 5% of the compressive strength of the spinal cage prior to sterilization.

Aspect 21. The spinal cage of any preceding aspect, further comprising one or more of a screw plate mated to the spinal cage, an insertion tool guide, or an engagement feature.

Aspect 22. The spinal cage of any preceding aspect, wherein the spinal cage is mated to a plate, an insertion tool guide, or an engagement feature.

Aspect 23. The spinal cage of any preceding aspect, wherein the polymer composition comprises less than 100 parts per million of halogen atoms.

Aspect 24. A spinal fusion system comprising: the spinal cage according to any of the preceding aspects and a plate, wherein the plate secures the spinal cage to the vertebrae.

Aspect 25. The spinal fusion system of aspect 24, wherein the plate comprises polyetherimide.

Aspect 26. A method of treating a spine of a patient comprising: removing a damaged spinal disk and inserting the spinal cage according to any of the previous aspects into an area of the spine that contained the damaged spinal disk, wherein the spinal cage is formed from a polyether ether ketone, a polyether ketone ketone, a polyarylsulphone, or a polyetherimide comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 27. A method of treating a spine of a patient consisting essentially of: removing a damaged spinal disk and inserting the spinal cage according to any of the previous aspects into an area of the spine that contained the damaged spinal disk, wherein the spinal cage is formed from a polyether ether ketone, a polyether ketone ketone, a polyarylsulphone, or a polyetherimide comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 28. A method of treating a spine of a patient consisting of: removing a damaged spinal disk and inserting the spinal cage according to any of the previous aspects into an area of the spine that contained the damaged spinal disk, wherein the spinal cage is formed from a polyether ether ketone, a polyether ketone ketone, a polyarylsulphone, or a polyetherimide comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 29. The method of any of aspects 26-28, wherein the polyetherimide has a weight average molecular weight of at least about 10,000 to about 150,000 grams per mole (g/mol).

Aspect 30. The method of any one of aspects 26-29, wherein the polyetherimide has less than 100 ppm amine end groups.

Aspect 31. The method of any one of aspects 26-30, further comprising a biocide, wherein the biocide is selected from germicides, antimicrobials, antibiotics, antibacterials, antiyeasts, antialgals, antivirals, antifungals, antiprotozoals, antiparasites, agents promoting bone or skeletal growth, and combinations thereof.

Aspect 32. The method of any one of aspects 26-31, wherein the spinal cage is formed from a polymer component comprising between 40 wt % and 90 wt % of the polyetherimide and between 10 wt % and 60 wt % of a filler by weight of the polymer component.

Aspect 33. The method of aspect 32, wherein the filler comprises glass, carbon, carbon fiber, or a combination thereof.

Aspect 34. The method of any one of aspects 26-33, wherein the polymer composition further comprises ceramic or metal.

Aspect 35. The method of any one of claims 26-34, wherein the input is custom data associated with a particular patient and the blank is formed using injection molding.

Aspect 36. The method of any one of claims 26-35, wherein the input is surface geometry of a patients interfacing vertebrae, protrusions, surface pores, registration features, dents, or other surfacing geometries.

Aspect 37. A spinal cage for implantation between two adjacent vertebrae, the spinal cage formed from a polymer composition comprising a polyetherimide, a polyether ether ketone, a polyether ketone ketone, the spinal cage formed from a process comprising: receiving an input relating to design specifications of the spinal cage; and causing formation of at least a portion of the spinal cage based upon the input and using one or more of an additive and subtractive process.

Aspect 38. A spinal cage for implantation between two adjacent vertebrae, the spinal cage formed from a polymer composition comprising a polyetherimide, a polyether ether ketone, a polyarylsulphone, or a polyether ketone ketone, or a combination thereof the spinal cage formed from a process consisting essentially of: receiving an input relating to design specifications of the spinal cage; and causing formation of at least a portion of the spinal cage based upon the input and using one or more of an additive and subtractive process.

Aspect 39. A spinal cage for implantation between two adjacent vertebrae, the spinal cage formed from a polymer composition comprising a polyetherimide, a polyether ether ketone, a polyether ketone ketone, a polyarylsulphone, or a combination thereof the spinal cage formed from a process consisting of: receiving an input relating to design specifications of the spinal cage; and causing formation of at least a portion of the spinal cage based upon the input and using one or more of an additive and subtractive process.

Aspect 40. The spinal cage of any of aspects 37-39, wherein the polyetherimide comprises structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 41. The spinal cage of any one of aspects 37-40, wherein the polyetherimide has a weight average molecular weight of at least about 10,000 to about 150,000 grams per mole (g/mol).

Aspect 42. The spinal cage of any one of aspects 37-41, wherein the polyetherimide has less than 100 ppm amine end groups.

Aspect 43. The spinal cage of any one of aspects 37-42, further comprising a biocide, wherein the biocide is selected from germicides, antimicrobials, antibiotics, antibacterials, antiyeasts, antialgals, antivirals, antifungals, antiprotozoals, antiparasites, agents promoting bone or skeletal growth, and combinations thereof.

Aspect 44. The spinal cage of any one of aspects 37-43, wherein the polymer composition further comprises ceramic or metal.

Aspect 45. The spinal cage of any one of aspects 37-44 wherein polyetherimide comprises repeating units of the formula

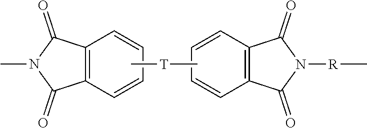

wherein R is a divalent radical of the formula

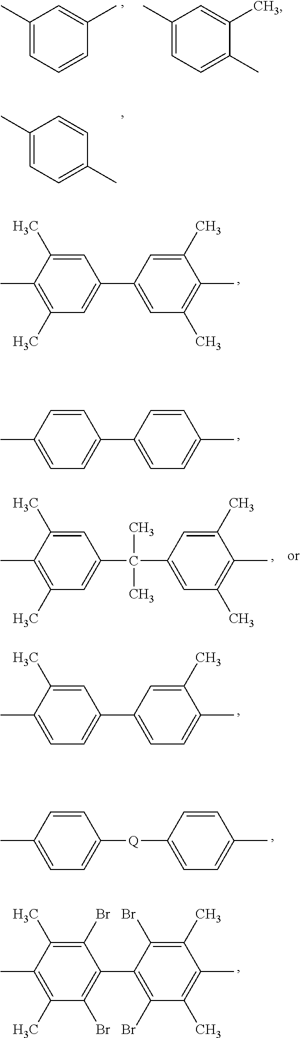

or combinations thereof wherein Q is selected from —O—, —S—, —C(O)—, —SO₂—, —SO—, and —C_{y}H_{2y}— wherein y is an integer from 1 to 5; and T is —O— or a group of the formula —O—Z—O— wherein the divalent bonds of the —O— or the —O—Z—O— group are in the 3,3′, 3,4′, 4,3′, or the 4,4′ positions and Z is a divalent group of the formula

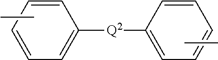

wherein Q² is selected from —O—, —S—, —C(O)—, —SO₂—, —SO—, and —C_{y}H_{2y}— wherein y is an integer from 1 to 5.

Aspect 46. The spinal cage according to any one of 37-45, further comprising sterilizing the spinal cage using at least one sterilization process selected from the group consisting of: steam autoclave sterilization, hydrogen peroxide sterilization, gamma-ray sterilization, electron beam radiation, and ethylene oxide sterilization.

Aspect 47. The spinal cage according to any one of aspects 37-46, wherein the spinal cage has a compressive strength after sterilization that is within 5% of the compressive strength of the spinal cage prior to sterilization.

Aspect 48. The spinal cage of any one of aspects 37-47, wherein the spinal cage comprises about 60 wt % to about 90 wt % base thermoplastic comprising polyetherimide and about 10 wt % to about 40 wt % filler material comprising carbon or glass.

Aspect 49. The spinal cage of any one of aspects 37-48, wherein the input is custom data associated with a particular patient and/or wherein the blank is formed using injection molding.

Aspect 50. The method of any one of claims 37-48, wherein the input is surface geometry of a patients interfacing vertebrae, protrusions, surface pores, registration features, dents, or other surfacing geometries.

Aspect 51. A method of making a spinal cage for implantation between two adjacent vertebrae, the spinal cage formed from a polymer composition comprising a polyetherimide, the method comprising: receiving an input relating to design specifications of the spinal cage; and applying a subtractive manufacturing process to a blank of the polymer composition to form at least a portion of the spinal cage based on the input.

Aspect 52. The method of aspect 51, wherein the polyetherimide comprises structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 53. The method of any one of aspects 51-52, wherein the polyetherimide has a weight average molecular weight of at least about 10,000 to about 150,000 grams per mole (g/mol).

Aspect 54. The method of any one of aspects 51-53, wherein the polyetherimide has less than 100 ppm amine end groups.

Aspect 55. The method according to any one of aspects 51-54, wherein the spinal cage has a compressive strength after sterilization that is within 5% of the compressive strength of the spinal cage prior to sterilization.

Aspect 56. The method of any one of aspects 51-55, wherein the spinal cage comprises about 60 wt % to about 90 wt % base thermoplastic comprising polyetherimide and about 10 wt % to about 40 wt % filler material comprising carbon or glass.

Aspect 57. The method of any one of aspects 51-56, wherein the input is custom data associated with a particular patient.

Aspect 58. The method of any one of claims 51-57, wherein the input is surface geometry of a patients interfacing vertebrae, protrusions, surface pores, registration features, dents, or other surfacing geometries.

Aspect 59. A method of making a spinal cage for implantation between two adjacent vertebrae, the spinal cage formed from a polymer composition comprising a polyetherimide, the method comprising: receiving an input relating to design specifications of the spinal cage associated with a particular patient; and processing the input to cause an additive manufacturing device to form at least a portion of the spinal cage.

Aspect 60. The method of aspect 59, wherein the polyetherimide comprises structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 61. The method of any one of aspects 59-60, wherein the polyetherimide has a weight average molecular weight of at least about 10,000 to about 150,000 grams per mole (g/mol).

Aspect 62. The method of any one of aspects 59-61, wherein the polyetherimide has less than 100 ppm amine end groups.

Aspect 63. The method according to any one of aspects 59-62, wherein the spinal cage has a compressive strength after sterilization that is within 5% of the compressive strength of the spinal cage prior to sterilization.

Aspect 64. The method of any one of aspects 59-63, wherein the spinal cage comprises about 60 wt % to about 90 wt % base thermoplastic comprising polyetherimide and about 10 wt % to about 40 wt % filler material comprising carbon or glass.

Aspect 65. The method of any one of aspects 59-64, wherein at least a portion of the spinal cage is formed using injection molding.

### Examples

As an illustrative example, the polyetherimides used in forming the apparatus of the present disclosure may exhibit distinguishable properties over other comparative polymers, as shown in Tables 2-3 (PEI—polyetherimide; PPSU—polyphenylsulfone; PSU—polysulfone; PEEK—Polyether ether ketone; TPU—thermoplastic polyurethane) as shown in Tables 2 and 3.

The following data apply: tensile stress was obtained in millimeters per minute (mm/min), kilogram-force centimeter (cm-kgf/cm), kilogram-force per square centimeter (kgf/cm²). Heat deflection temperature (HDT) in megapascals (MPa). Volume resistivity is presented in Ohm-centimeters (Ohm·cm).

TABLE 2

| | | | E1 | E2 | CE1 | CE2 | CE3 |
|---|---|---|---|---|---|---|---|
| Polymer Type | | | PEI-1 | PEI-2 | PPSU | PSU | PEEK |
| MECHANICAL | Unit | Standard | | | | | |
| Tensile Stress at | kilogram | ASTM | 1120 | 1120 | 710 | 720 | 1020 |
| Yield, Type I, 5 mm/min | force · meter | D 638 | | | | | |
| | kgf/cm² | | | | | | |
| Tensile Modulus, | kgf/cm² | ASTM | 36500 | 36500 | 23900 | 25300 | 37700 |
| 5 mm/min | | D 638 | | | | | |
| Flexural Stress at | kgf/cm² | ASTM | 1760 | 1770 | 930 | 1080 | 1560 |
| Yield, 1.3 mm/min, | | D 790 | | | | | |
| 50 mm | | | | | | | |
| span | | | | | | | |
| Flexural | kgf/cm² | ASTM | 35000 | 34900 | 24600 | 27400 | 38700 |
| Modulus, 1.3 mm/min, | | D 790 | | | | | |
| 50 mm | | | | | | | |
| span | | | | | | | |
| IMPACT | Unit | Standard | Value | | | | |
| Izod Impact, | cm- | ASTM | 5 | 3 | 70 | 7.0 | 5.4 |
| notched, 23° C. | kgf/cm | D 256 | | | | | |
| PHYSICAL | Unit | Standard | | Value | | | |
| Specific Gravity | — | ASTM | 1.27 | 1.27 | 1.29 | 1.24 | 1.30 |
| | | D 792 | | | | | |
| Melt Flow Rate, | g/10 min | ASTM | — | — | — | — | 36 |
| 400° C./2.16 kgf | | D 1238 | | | | | |
| Melt Flow Rate, | g/10 min | ASTM | — | — | 14-20 | — | — |
| 365° C./5.0 kgf | | D 1238 | | | | | |
| Melt Flow Rate, | g/10 min | ASTM | — | — | — | 6.5 | — |
| 343° C./2.16 kgf | | D 1238 | | | | | |
| Melt Flow Rate, | g/10 min | ASTM | 9 | 17.8 | — | — | — |
| 337° C./6.6 kgf | | D 1238 | | | | | |
| ELECTRICAL | Unit | Standard | | Value | | | |
| Volume | Ohm-cm | ASTM | 1.00E+17 | 1.00E+17 | 9.00E+15 | 3.00E+16 | — |
| Resistivity | | D 257 | | | | | |
| THERMAL | Unit | Standard | | Value | | | |
| Glass Transition | ° C. | | 217 | 217 | 220 | — | 147 |
| Temperature | | | | | | | |
| Heat Deflection | ° C. | ASTM | 201 | 198 | 207 | 174 | 160 |
| Temperature, | | D 648 | | | | | |
| 1.82 MPa | | | | | | | |

TABLE 3

| | | | E1 | E2 | CE4 | CE5 | CE6 |
|---|---|---|---|---|---|---|---|
| Polymer Type | | | PEI-I | PEI-2 | TPU | TPU | TPU |
| MECHANICAL | Unit | Standard | | | | | |
| Tensile Stress at Yield, | kgf/cm² | ASTM | — | — | — | 720 | 1020 |
| Type I, 5 mm/min | | D 638 | | | | | |
| Tensile Modulus, 5 mm/min | kgf/cm² | ASTM | — | — | — | 25300 | 37700 |
| | | D 638 | | | | | |
| Flexural Stress at Yield, | kgf/cm² | ASTM | 16 | 63 | 770 | 1080 | 1560 |
| 1.3 mm/min, 50 mm span | | D 790 | | | | | |
| Flexural Modulus, 1.3 mm/min, | kgf/cm² | ASTM | 370 | 1520 | 20320 | 27400 | 38700 |
| 50 mm span | | D 790 | | | | | |
| IMPACT | Unit | Standard | | | | | |
| Izod Impact, notched, | cm- | ASTM | — | — | — | 7.0 | 5.4 |
| 23° C. | kgf/cm | D 256 | | | | | |
| PHYSICAL | Unit | Standard | | | | | |
| Specific Gravity | — | ASTM | 1.12 | 1.16 | 1.19 | 1.24 | 1.30 |
| | | D 792 | | | | | |
| Melt Flow Rate, | g/10 min | ASTM | 9 | 17.8 | — | — | — |
| 337° C./6.6 kgf | | D 1238 | | | | | |
| Melt Flow Rate, 224° C. | g/10 min | ASTM | — | — | 17 | 13 | 37 |
| | | D 1238 | | | | | |
| ELECTRICAL | Unit | Standard | | | | | |
| Volume Resistivity | Ohm- | ASTM | — | — | — | 3.00E+16 | — |
| | cm | D 257 | | | | | |
| THERMAL | Unit | Standard | | | | | |
| Glass Transition | ° C. | | — | — | — | — | 147 |
| Temperature | | | | | | | |
| Heat Deflection | ° C. | ASTM | — | — | — | 174 | 160 |
| Temperature, 1.82 MPa | | D 648 | | | | | |

As shown in Tables 2 and 3, inventive examples E1 and E2 demonstrate comparable physical and mechanical properties to those observed for comparative examples CE1 through CE6 comprising a range of polymers.

It will be appreciated that the foregoing description provides examples of the disclosed system and technique. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

1. A spinal cage for implantation between two adjacent vertebrae, the spinal cage formed from a polymer composition, the spinal cage formed from a process comprising:
a. receiving an input relating to design specifications of the spinal cage; and
b. causing formation of at least a portion of the spinal cage based upon the input and using an additive manufacturing process, a subtractive manufacturing process or a combination thereof at a spinal cage core,
wherein the spinal cage core is a standardized pre-manufactured spinal cage core.

2. The spinal cage of claim 1, wherein the polymer composition comprises a biocompatible polymer.

3. The spinal cage of claim 1, wherein the polymer composition comprises a polyether ether ketone.

4. The spinal cage of claim 1, further comprising a biocide, wherein the biocide is selected from germicides, antimicrobials, antibiotics, antibacterials, antiyeasts, antialgals, antivirals, antifungals, antiprotozoals, antiparasites, and combinations thereof.

5. The spinal cage of claim 1, wherein the polymer composition further comprises ceramic or metal.

6. The spinal cage of claim 1, wherein the polymer composition comprises a polyetherimide comprises comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

7. The spinal cage according to claim 1, further comprising sterilizing the spinal cage using at least one sterilization process selected from the group consisting of: steam autoclave sterilization, hydrogen peroxide sterilization, gamma-ray sterilization, electron beam radiation, and ethylene oxide sterilization.

8. The spinal cage according to claim 1, wherein the spinal cage has a compressive strength after sterilization that is within 5% of the compressive strength of the spinal cage prior to sterilization.

9. The spinal cage of claim 1, wherein the spinal cage comprises about 60 wt. % to about 90 wt. % of the polymer composition and about 10 wt % to about 40 wt % filler material comprising carbon or glass.

10. The spinal cage of claim 1, wherein the input is custom data associated with a particular patient.

11. The spinal cage of claim 1, further comprising one or more of a screw plate mated to the spinal cage, an insertion tool guide, or an engagement feature.

12. The spinal cage of claim 1, wherein the polymer composition comprises less than 100 parts per million of halogen atoms.

13. A method of making a spinal cage for implantation between two adjacent vertebrae, the spinal cage formed from a polymer composition, the method comprising:
a. receiving an input relating to design specifications of the spinal cage; and
b. applying a subtractive manufacturing process to a blank of the polymer composition to form at least a portion of a spinal cage based on the input,
wherein the blank is a standardized spinal cage core.

14. The method of claim 13, wherein the polymer composition comprises polyetherimide comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

15. The method according to claim 13, wherein the spinal cage has a compressive strength after sterilization that is within 5% of the compressive strength of the spinal cage prior to sterilization.

16. The method of claim 13, wherein the spinal cage comprises about 60 wt % to about 90 wt % polymer composition comprising polyetherimide, polyether ether ketone, or poly ether ketone ketone, and about 10 wt % to about 40 wt % filler material comprising carbon or glass.

17. The method of claim 13, further comprising sterilizing the spinal cage using at least one sterilization process selected from the group consisting of: steam autoclave sterilization, hydrogen peroxide sterilization, gamma-ray sterilization, electron beam radiation, and ethylene oxide sterilization.

18. The method of claim 13, wherein the input is custom data associated with a particular patient and the blank is formed using injection molding.

19. A method of making a spinal cage for implantation between two adjacent vertebrae, the spinal cage formed from a polymer composition, the method comprising:
a. receiving an input relating to design specifications of the spinal cage associated with a particular patient; and
b. processing the input to cause an additive manufacturing device to form at least a portion of the spinal cage.

20. The method of claim 19, wherein at least a portion of the spinal cage is formed using injection molding.
